(19) United States
(12) Patent Application Publication
(10) Pub. No.: US 2026/0224361 A1
(43) Pub. Date: Aug. 6, 2026

(54) OUTER SKIRTS FOR PROSTHETIC HEART VALVES AND ASSOCIATED METHODS

(71) Applicant: Edwards Lifesciences Corporation, Irvine, CA (US)

(72) Inventor: Tamir S. Levi, Zikhron Yaakov (IL)

(73) Assignee: Edwards Lifesciences Corporation, Irvine, CA (US)

(21) Appl. No.: 19/639,663

(22) Filed: Apr. 6, 2026

Related U.S. Application Data
(63) Continuation of application No. PCT/US2024/051367, filed on Oct. 15, 2024.
(60) Provisional application No. 63/544,264, filed on Oct. 16, 2023.

(51) Int. Cl. A61F 2/24; A61L 27/34; A61L 33/00

(52) U.S. Cl. CPC A61F 2/2418; A61L 27/34; A61L 33/0011; A61F 2230/0006; A61F 2250/0021; A61F 2250/0035; A61F 2250/0067; A61L 2400/04

(57) ABSTRACT

Prosthetic heart valves including an annular frame and an outer skirt and methods associated therewith are disclosed. The outer skirt is disposed on an outer surface of the frame. In some examples, the outer skirt a plurality of higher friction sections and a plurality of lower friction sections axially extending between inflow and outflow ends of the outer skirt and having an alternating arrangement around a circumference of the outer skirt. In a radially compressed state of a prosthetic valve, the higher friction sections are folded, forming interior folds, and the lower friction sections form exterior folds extending over and shielding the interior folds. In a radially expanded state of the prosthetic valve, the interior folds are unfolded, so that the higher friction sections are exposed on the exterior surface of the prosthetic valve. In some examples, a prosthetic valve includes one or more treated or coated surfaces or layers.

### CROSS-REFERENCE TO RELATED APPLICATIONS

This application is a continuation of PCT Application No. PCT/US2024/051367, filed October 15, 2024, which claims the benefit of U.S. Provisional Patent Application No. 63/544,264, filed October 16, 2023, the entire contents of each of which are incorporated herein by reference.

### FIELD

The present disclosure relates to prosthetic heart valves, including a frame, a valve structure disposed on an interior of the frame, and an outer skirt disposed on an exterior of the frame, as well as methods of implantation thereof.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve includes a frame, a valve structure disposed on an interior of the frame, and an outer skirt disposed on an exterior of the frame.

The prosthetic valve can be mounted in a crimped state on the distal end of a delivery apparatus and advanced through an introducer sheath and the patient’s vasculature (for example, through a femoral artery and the aorta) until the prosthetic heart valve reaches the implantation site (for example, a native valve) in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

After expansion to its functional size, the outer skirt contacts tissue of the native valve and/or the surrounding anatomy and forms a seal between the prosthetic valve and the tissue, and the valve structure regulates flow of blood through the valve.

### SUMMARY

Described herein are prosthetic heart valves, delivery apparatus, and methods for implanting prosthetic heart valves. The disclosed prosthetic heart valves, delivery apparatus, and methods can, for example, provide reduced friction between a prosthetic heart valve and components of a delivery apparatus for smoother and easier (for example, requiring less force) delivery of the prosthetic valve. Additionally, the prosthetic heart valves can encourage tissue ingrowth between tissue at an implantation site and an exterior surface of an outer skirt of the prosthetic valve for stabilization and/or sealing of the prosthetic valve after implantation. As such, the devices and methods disclosed herein can, among other things, overcome one or more of the deficiencies of typical prosthetic heart valves and their delivery apparatus.

A prosthetic heart valve can comprise a frame and a valve structure coupled to the frame. In addition to these components, a prosthetic heart valve can further comprise one or more of the components disclosed herein.

In some examples, a prosthetic heart valve can comprise an outer skirt.

In some examples, an outer skirt can include a plurality of lower friction sections and a plurality of higher friction sections.

In some examples, in a radially compressed state of the prosthetic heart valve, the higher friction sections can form a plurality of interior folds and the lower friction sections can form a plurality of exterior folds.

In some examples, the plurality of exterior folds of the outer skirt extend at least partially over the interior folds such that at least a portion of the total surface area of the higher friction sections is covered by the lower friction sections while the prosthetic heart valve in the radially compressed state.

In some examples, the plurality of exterior folds are configured to unfold as the valve transitions from the radially compressed state to a radially expanded state.

In some examples, in the radially expanded state of the prosthetic heart valve, a lesser extent of the total surface area of the higher friction sections is covered by the lower friction sections relative to when the prosthetic heart valve is in the radially compressed state.

In some examples, when the prosthetic heart valve is in the radially compressed state, the higher friction sections are completely covered by the lower friction sections, and wherein, when the prosthetic heart valve is in the radially expanded state, the higher friction sections are completely uncovered by the lower friction sections.

In some examples, an outer skirt includes a plurality of lower friction sections and a plurality of higher friction sections having an alternating arrangement around a circumference of the outer skirt.

In some examples, the lower friction section and the higher friction sections extend axially from an inflow end of the outer skirt to an outflow end of the outer skit.

In some examples, the higher friction sections comprise a textured material, such as, for example, a pile material, a plush material, or a fringed material.

In some examples, the lower friction sections comprise a smoother material relative to the textured material.

In some examples, the higher friction sections have a greater total surface area than the lower friction sections.

In some examples, a ratio of a total surface area of the lower friction sections relative to a total surface area of higher friction sections is in a range of 0.2-0.4.

In some examples, an outer skirt comprises a base layer having textured material attached thereto or formed thereon to create higher friction sections, while exposed portions of the base layer are devoid of the textured material and form lower friction sections.

In some examples, an outer skirt can include a first surface treatment or coating on the lower friction sections and a second surface treatment or coating on the higher friction sections.

In some examples, the lower friction sections comprise a hydrophilic or lubricious surface treatment or coating.

In some examples, the higher friction sections comprise a prothrombogenic surface treatment or coating.

In some examples, an outer skirt can include a base layer comprised of an impermeable or semi-impermeable material.

In some examples, an outer skirt can include a first agent-eluting layer on an interior surface of the base layer, and a second agent-eluting layer on an exterior surface of the base layer.

In some examples, the first agent-eluting layer on the interior surface includes an antithrombogenic coating or treatment.

In some examples, the second agent-eluting layer on the exterior surface of the base layer includes a prothrombogenic coating or treatment.

In some examples, the second agent-eluting layer on the exterior surface of the base layer includes a lubricous coating or treatment.

In some examples, an outer skirt includes a polyethylene glycol (PEG) treatment on an outer surface thereof.

In some examples, the PEG is elutable from the outer skirt upon contact with fluid.

In some examples, a prosthetic heart valve comprises: an annular frame having an inflow end and an outflow end, wherein the annular frame is radially expandable for transitioning the prosthetic heart valve from a radially compressed state to a radially expanded state; a leaflet structure positioned within an interior of the frame; and an outer skirt positioned on an exterior of the frame, wherein an exterior surface of the outer skirt comprises a plurality of lower friction sections and a plurality of higher friction sections; wherein, in the radially compressed state of the prosthetic heart valve, the higher friction sections form a plurality of interior folds of the outer skirt and the lower friction sections form a plurality of exterior folds of the outer skirt that extend at least partially over the interior folds such that at least a portion of a total surface area of the higher friction sections is covered by the lower friction sections, and in the radially expanded state of the prosthetic heart valve, a lesser extent of the total surface area of the higher friction sections is covered by the lower friction sections..

In some examples, a prosthetic heart valve comprises: an annular frame having an inflow end and an outflow end, wherein the annular frame is radially expandable for transitioning the prosthetic heart valve from a radially compressed state to a radially expanded state; a leaflet structure positioned within an interior of the frame; and an outer skirt positioned on an exterior of the frame, wherein an exterior surface of the outer skirt comprises a plurality of lower friction sections and a plurality of higher friction sections; wherein, in the radially compressed state of the prosthetic heart valve, the higher friction sections form a plurality of interior folds of the outer skirt and the lower friction sections extend at least partially over the interior folds; and wherein, when the prosthetic heart valve transitions from the radially compressed state to the radially expanded state, the higher friction sections unfold such that a greater portion of the higher friction sections are uncovered from the lower friction sections relative to when the prosthetic heart valve is in the radially compressed state.

In some examples, a prosthetic heart valve comprises: an annular frame having an inflow end and an outflow end; a leaflet structure positioned within an interior of the frame; and an outer skirt positioned on an exterior of the frame and secured to the frame, wherein the outer skirt comprises an impermeable or semi-impermeable layer, an interior agent-eluting layer, and an exterior agent-eluting layer.

In some examples, a prosthetic heart valve comprises one or more of the components recited in Examples 1-43 below.

An assembly can comprise a prosthetic heart valve and a delivery apparatus.

In some examples, the prosthetic heart valve is radially compressible and balloon expandable.

In some examples, the prosthetic heart valve is radially compressible and self-expandable.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side elevation view of a prosthetic heart valve, according to one example.

FIG. 2 is a side view of an example of a delivery apparatus configured to deliver and implant a radially expandable prosthetic heart valve at an implantation site.

FIG. 3A is a cross-sectional view of exemplary prosthetic heart valve illustrating a first exemplary fold configuration or arrangement for an outer skirt when the prosthetic valve is in a radially compressed state, in accordance with the present disclosure.

FIG. 3B is a cross-sectional view of exemplary prosthetic heart valve illustrating a second exemplary fold configuration or arrangement for an outer skirt when the prosthetic valve is in a radially compressed state, in accordance with the present disclosure.

FIG. 4A is a perspective view of the outer skirt of the prosthetic heart valve of FIGS. 3A and 3B in an unfolded state (corresponding to a radially expanded state of the prosthetic heart valve).

FIG. 4B is a cross-sectional view of a portion of the outer skirt in the unfolded state of FIG. 4A.

FIG. 5 is a cross-sectional view of another exemplary outer skirt that can be utilized with a prosthetic heart valve.

FIG. 6 is a schematic, cross-sectional view of a first delivery apparatus configured for use with the prosthetic heart valves disclosed herein.

FIG. 7 is a schematic, cross-sectional view of a second delivery apparatus configured for use with the prosthetic heart valves disclosed herein.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

### Overview

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

In some examples, the prosthetic valves described herein can include an outer skirt with higher friction sections and lower friction sections. In some examples, the prosthetic valves described herein can include an outer skirt with one or more surface treatments or coatings or one or more agent-eluting surfaces.

### Examples of the Disclosed Technology

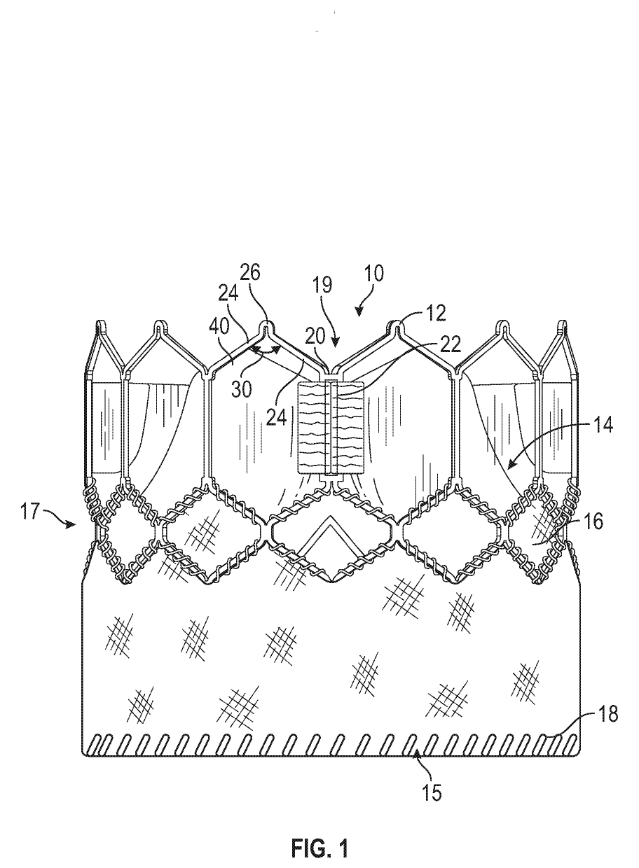

FIG. 1 shows an exemplary prosthetic valve 10, according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 comprises four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19. The inner skirt 16 can be arranged on and/or coupled to an inner surface of the frame 12, while the outer skirt 18 can be arranged on and/or coupled to an outer surface of the frame 12.

One or more of the skirts 16, 18 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plush nap or pile include velour, velvet, velveteen, corduroy, terrycloth, fleece, etc. In some examples, the skirt can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

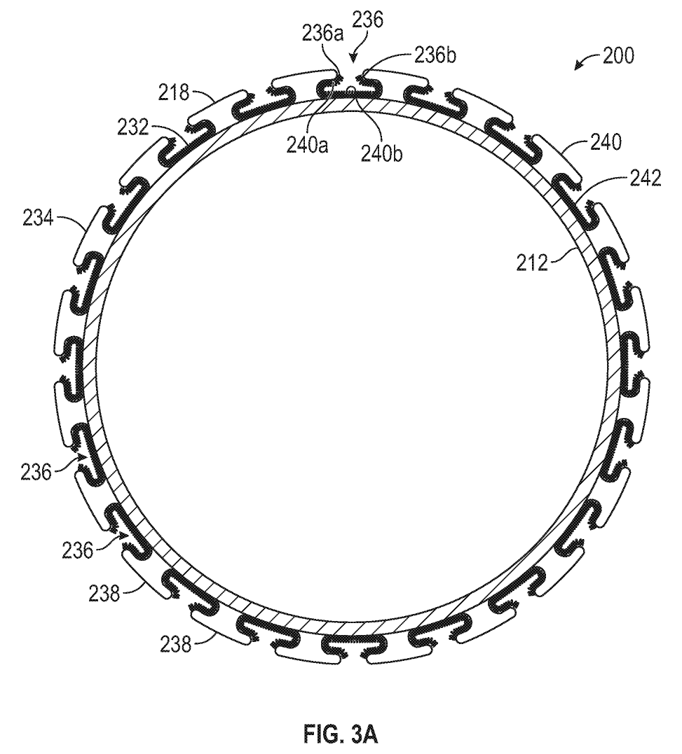

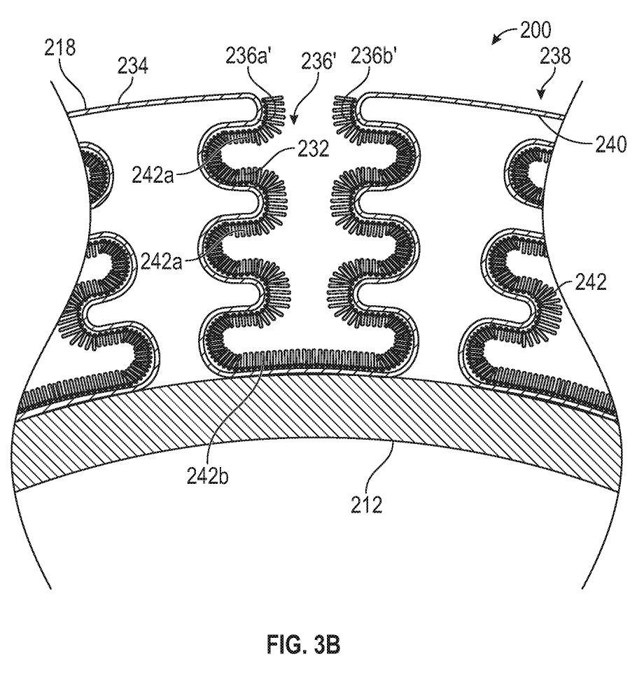

In some examples, the outer skirt can include a combination of the foregoing materials. For example, an outer skirt can include a non-textile or film base layer having one or more sections of fabric material (such as, a plush or pile material) attached thereto or formed thereon. Exemplary outer skirts including a combination of materials are discussed further below with reference to FIGS. 3A-4B.

The valvular structure 14 can comprise three leaflets 40, collectively forming a leaflet structure, which can be arranged to collapse in a tricuspid arrangement, although in other examples there can be greater or fewer number of leaflets (for example, one or more leaflets 40). The leaflets 40 can be secured to one another at their adjacent sides to form commissures 22 of the leaflet structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein. As described in more detail below, in some examples, the leaflets 40 can be folded to form neo-commissures that are circumferentially offset from the commissures 22. In these examples, the folding pattern of the leaflets 40 can enable the leaflets to be self-adjustable such that the leaflets 40 are operable to regulate blood flow at each diameter within a range of diameters.

The frame 12 can be radially compressible (collapsible) and expandable (for example, expanded configuration shown in FIG. 1) and comprise a plurality of interconnected struts 24. A plurality of apices 26 that are spaced circumferentially apart are formed at the inflow end portion 15 and the outflow end portion 19 of the frame 12 (only the apices 26 at the outflow end portion 19 are visible in FIG. 1). Each apex 26 is formed at a junction between two angled struts 24 at either the inflow end portion 15 or the outflow end portion 19. FIG. 1 depicts a known frame design with apices 26 that form a U-shaped bend between the two angled struts 24. In some examples, an angle 30 between the two angled struts 24, connected at the apex 26, can be in a range of 90 to 120 degrees.

The frame 12 can be formed with a plurality of circumferentially spaced slots, or commissure windows 20 that are adapted to mount the commissures 22 of the valvular structure 14 to the frame. The frame 12 can be made of any of various suitable plastically expandable materials (for example, stainless steel, etc.) or shape-memory, self-expanding materials (for example, Nitinol). When constructed of a plastically expandable material, the frame 12 (and thus the prosthetic valve 10) can be crimped to a radially collapsed configuration on a delivery catheter or apparatus and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

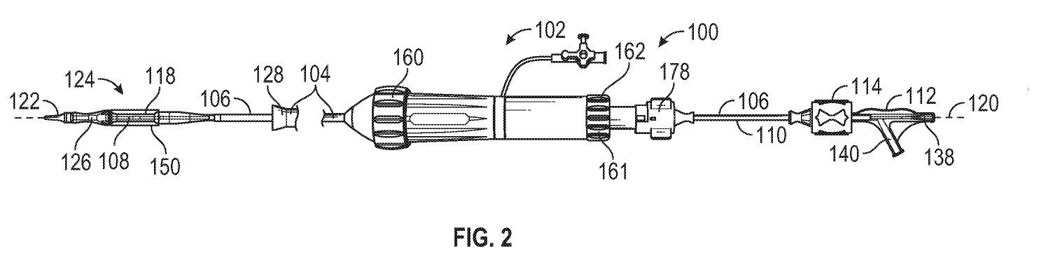

The frame 12 can be made of any of various suitable plastically-expandable materials (for example, stainless steel, etc.) or self-expanding materials (for example, Nitinol) as known in the art. When constructed of a plastically-expandable material, the frame 12 (and thus the valve 10) can be crimped to a radially compressed state on a delivery catheter and then expanded inside a patient by an inflatable balloon or equivalent expansion mechanism. When constructed of a self-expandable material, the frame 12 (and thus the valve 10) can be crimped to a radially compressed state and restrained in the compressed state by insertion into a sheath or equivalent mechanism of a delivery catheter (such as, the delivery apparatus 100 shown in FIG. 2 and discussed below). Once inside the body, the valve 10 can be advanced from the delivery sheath, which allows the valve to expand to its functional size.

Suitable plastically-expandable materials that can be used to form the frames disclosed herein (for example, the frame 12) include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Additional details regarding the prosthetic valve 10 and its various components are described in WIPO Patent Application Publication No. WO 2018/222799, which is incorporated herein by reference.

FIG. 2 shows a delivery apparatus 100, according to an example, that can be used to implant an expandable prosthetic heart valve (for example, the prosthetic heart valve 10 of FIG. 1 and/or any of the other prosthetic heart valves described herein). In some examples, the delivery apparatus 100 is specifically adapted for use in introducing a prosthetic valve into a heart.

The delivery apparatus 100 in the illustrated example of FIG. 2 is a balloon catheter comprising a handle 102 and a steerable, outer shaft 104 extending distally from the handle 102. The delivery apparatus 100 can further comprise an intermediate shaft 106 (which also may be referred to as a balloon shaft) that extends proximally from the handle 102 and distally from the handle 102, the portion extending distally from the handle 102 also extending coaxially through the outer shaft 104. Additionally, the delivery apparatus 100 can further comprise an inner shaft 108 extending distally from the handle 102 coaxially through the intermediate shaft 106 and the outer shaft 104 and proximally from the handle 102 coaxially through the intermediate shaft 106.

The outer shaft 104 and the intermediate shaft 106 can be configured to translate (for example, move) longitudinally, along a central longitudinal axis 120 of the delivery apparatus 100, relative to one another to facilitate delivery and positioning of a prosthetic valve at an implantation site in a patient’s body.

The intermediate shaft 106 can include a proximal end portion 110 that extends proximally from a proximal end of the handle 102, to an adaptor 112. A rotatable knob 114 can be mounted on the proximal end portion 110 and can be configured to rotate the intermediate shaft 106 around the central longitudinal axis 120 and relative to the outer shaft 104.

The adaptor 112 can include a first port 138 configured to receive a guidewire therethrough and a second port 140 configured to receive fluid (for example, inflation fluid) from a fluid source. The second port 140 can be fluidly coupled to an inner lumen of the intermediate shaft 106.

The intermediate shaft 106 can further include a distal end portion that extends distally beyond a distal end of the outer shaft 104 when a distal end of the outer shaft 104 is positioned away from an inflatable balloon 118 of the delivery apparatus 100. A distal end portion of the inner shaft 108 can extend distally beyond the distal end portion of the intermediate shaft 106.

The balloon 118 can be coupled to the distal end portion of the intermediate shaft 106.

In some examples, a distal end of the balloon 118 can be coupled to a distal end of the delivery apparatus 100, such as to a nose cone 122 (as shown in FIG. 2), or to an alternate component at the distal end of the delivery apparatus 100 (for example, a distal shoulder). An intermediate portion of the balloon 118 can overlay a valve mounting portion 124 of a distal end portion of the delivery apparatus 100 and a distal end portion of the balloon 118 can overlay a distal shoulder 126 of the delivery apparatus 100. The valve mounting portion 124 and the intermediate portion of the balloon 118 can be configured to receive a prosthetic heart valve in a radially compressed state. For example, as shown schematically in FIG. 2, a prosthetic heart valve 150 (which can be one of the prosthetic valves described herein) can be mounted around the balloon 118, at the valve mounting portion 124 of the delivery apparatus 100.

The balloon shoulder assembly, including the distal shoulder 126, is configured to maintain the prosthetic heart valve 150 (or other medical device) at a fixed position on the balloon 118 during delivery through the patient’s vasculature.

The outer shaft 104 can include a distal tip portion 128 mounted on its distal end. The outer shaft 104 and the intermediate shaft 106 can be translated axially relative to one another to position the distal tip portion 128 adjacent to a proximal end of the valve mounting portion 124, when the prosthetic valve 150 is mounted in the radially compressed state on the valve mounting portion 124 (as shown in FIG. 2) and during delivery of the prosthetic valve to the target implantation site. As such, the distal tip portion 128 can be configured to resist movement of the prosthetic valve 150 relative to the balloon 118 proximally, in the axial direction, relative to the balloon 118, when the distal tip portion 128 is arranged adjacent to a proximal side of the valve mounting portion 124.

An annular space can be defined between an outer surface of the inner shaft 108 and an inner surface of the intermediate shaft 106 and can be configured to receive fluid from a fluid source via the second port 140 of the adaptor 112. The annular space can be fluidly coupled to a fluid passageway formed between the outer surface of the distal end portion of the inner shaft 108 and an inner surface of the balloon 118. As such, fluid from the fluid source can flow to the fluid passageway from the annular space to inflate the balloon 118 and radially expand and deploy the prosthetic valve 150.

An inner lumen of the inner shaft can be configured to receive a guidewire therethrough, for navigating the distal end portion of the delivery apparatus 100 to the target implantation site.

The handle 102 can include a steering mechanism configured to adjust the curvature of the distal end portion of the delivery apparatus 100. In the illustrated example, for example, the handle 102 includes an adjustment member, such as the illustrated rotatable knob 160, which in turn is operatively coupled to the proximal end portion of a pull wire. The pull wire can extend distally from the handle 102 through the outer shaft 104 and has a distal end portion affixed to the outer shaft 104 at or near the distal end of the outer shaft 104. Rotating the knob 160 can increase or decrease the tension in the pull wire, thereby adjusting the curvature of the distal end portion of the delivery apparatus 100. Further details on steering or flex mechanisms for the delivery apparatus can be found in U.S. Patent No. 9,339,384, which is incorporated by reference herein.

The handle 102 can further include an adjustment mechanism 161 including an adjustment member, such as the illustrated rotatable knob 162, and an associated locking mechanism including another adjustment member, configured as a rotatable knob 178. The adjustment mechanism 161 is configured to adjust the axial position of the intermediate shaft 106 relative to the outer shaft 104 (for example, for fine positioning at the implantation site). Further details on the delivery apparatus 100 can be found in PCT Application No. PCT/US2021/047056, which is incorporated by reference herein.

Turning to FIGS. 3A-4B, an exemplary prosthetic valve 200 in accordance with the present disclosure is shown and described. FIGS. 3A-3B show prosthetic valve 200 in a radially compressed state or configuration, and FIGS. 4A-4B show the prosthetic valve 200 in a radially expanded state or configuration. As can be seen in FIG. 3A, the prosthetic valve can include a frame 212 and an outer skirt 218 arranged on and/or coupled to an outer surface of the frame 212. In some examples, the frame 212 can have one or more of the features described above with respect to frame 12, or can have other features or configurations. Although not shown in FIGS. 3A-4B for clarity, the prosthetic valve 200 can further include a valvular structure (for example, a valvular structure having one or more of the features of the valvular structure 14 or other features or configurations) and/or an inner skirt (for example, an inner skirt having one or more features of the inner skirt 16 or other features or configurations).

As illustrated in FIGS. 3A-4B, the outer skirt 218 can include a plurality of first sections 232 and a plurality of second sections 234. In some examples, the first sections 232 are higher frictions sections and the second sections 234 are lower friction sections. Thus, the higher friction sections 232 can comprise, for example, a material or surface texture having a higher coefficient of friction relative to the lower friction sections 234, which can comprise, for example, a material or surface texture having a relatively lower coefficient of friction. In some examples, the higher friction sections 232 and the lower friction sections 234 can extend axially from an inflow end 215 of the outer skirt to an outflow end 219 of the outer skirt (FIG. 4A). In other examples, one or more of sections 232 and/or 234 do not extend the entire of the skirt and/or can different heights relative to each other. In some examples, the higher friction sections 232 and the lower friction sections 234 can have an alternating arrangement around a circumference of the outer skirt 218. In other words, the higher friction sections 232 can be circumferentially spaced apart from each other by having the lower frictions sections 234 inserted or disposed therebetween.

As discussed above, in some examples, an outer skirt, such as the outer skirt 218, can include a combination of materials. For example, the higher friction sections 232 can comprise a first material or first combination of materials, and the lower frictions sections 234 can comprise a second material or a second combination of materials different from the first material or first combination of materials. In some examples, the material(s) of the higher friction sections 232 can be textured and/or have a greater thickness than the material(s) of the lower friction sections 234, which can be smoother or have less texture and/or be thinner that the higher friction sections 232.

In some examples, the lower friction sections 234 can comprise a lower friction fabric, such as a tight weave, braided, or knitted fabric. In some examples, the lower friction fabric can include a low friction coating, such as polymeric material coating. In some examples, the lower friction sections 232 can comprise a fabric without interlaced yarns or fibers, such as a lower friction or smoother felt or electrospun fabric. In other examples, the lower friction sections can comprise a film or a sheet of a lower friction material, such as a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In other examples, the lower friction sections 234 can comprise natural tissue.

In some examples, the higher friction sections 232 can comprise a higher friction fabric, such as a loose weave, braided or knitted fabric or a plush nap or a pile material (such as, for example, velour, velvet, velveteen, corduroy, terrycloth, fleece, etc.). In some examples, the higher friction sections 234 can comprise a fabric without interlaced yarns or fibers or with randomly interlaced yarns or fibers, such as a higher friction or rougher felt or electrospun fabric. In other examples, the higher frictions sections 234 can comprise a sponge material or foam, such as polyurethane foam. In some examples, the higher friction sections comprise a plurality of fibers extending radially outward from an outer surface of the outer skirt.

In some examples, the higher friction sections can comprise a material that encourages tissue ingrowth. In some examples, each of the higher friction surfaces 232 and lower friction surfaces 234 can be separately formed and attached to each other, such as by stitching together the higher friction surfaces 232 and lower friction surfaces 234 along their adjacent edges.

In some examples, such as those shown in FIGS. 3A-4B, the lower friction sections 234 are formed by a base layer 240, such as a film or a sheet of a lower friction material, and the higher friction sections 232 are formed by a higher friction material 242, such as a plush or a pile material, a foam material, etc., attached to, formed on, or extruded from the base layer. In other words, each of the higher friction sections 234 can be discrete portion of a higher friction or textured material 242, while each of the lower friction sections 232 can be an exposed portion of the base layer 240 that is devoid of the higher friction or textured material 242. In some examples, the base layer 240 can be an impermeable or semi-impermeable layer that can limit or prevent elution or permeation of substances or agents from the interior of the prosthetic valve 200 to the exterior of the valve and/or from the exterior of the prosthetic valve 200 to the interior of the valve (discussed further below with reference to FIG. 4B).

In some examples, each of the higher friction sections 232 can have a greater surface area than each of the lower friction sections 234. For example, as shown in FIG. 4A, each of the higher friction sections 232 has a height a extending from the inflow end 215 to the outflow end 219 of the outer skirt and a width b extending from a first lateral edge of to a second lateral edge thereof, and each of the lower friction sections 234 can have a height a and a width c extending from a first lateral edge of to a second lateral edge thereof. The width b can be greater than the width c. For example, the width b can be in a range of 2 to 4 times greater that the width c. Accordingly, in such examples, a surface area of each of the higher friction sections (a x b) can be greater (such as in a range of 2 to 4 times greater) than a surface area of the each of the lower friction sections (a x c).

In some examples, the higher friction sections 232 can form a greater portion of the exterior surface area of the outer skirt than the lower friction sections 234. For example, the total surface area of the higher friction sections can be in a range of 60% to 80 % of the total surface area of the exterior surface of the outer skirt, whereas the total surface area of the lower friction sections can be in a range of 20% to 40% of the total surface area of the exterior surface of the outer skirt. In other words, a ratio of a total surface area of the lower friction sections relative to a total surface area of higher friction sections can be in a range of 0.2-0.4.

As can be seen in FIG. 3A-3B, in the radially compressed state of the prosthetic valve 200, the higher friction sections 232 can be folded inward and form a plurality of interior folds 236 of the outer skirt, while the lower friction sections 234 form a plurality of exterior folds 238. In some examples, the lower friction sections 234 forming the exterior folds 238 extend at least partially over the higher friction sections 232 forming the interior folds 236 such that at least a portion of the total surface area of the higher friction sections is covered by the lower friction sections. In other words, the higher friction sections 232 can be completely or partially covered or shielded by the lower friction sections 234 while the prosthetic valve 200 is radially compressed. Further, the lower friction sections 234 can form at least the majority of the outer surface of the prosthetic valve when the while the prosthetic valve 200 is radially compressed.

In some examples, such as in the example shown in FIG. 3A, each of the interior folds 236 can be formed by folding opposing end portions of a higher friction section 232 towards a center of the higher friction section 232. In such examples, each of the folds 236 includes a first portion 236a and a second portion 236b, and each portion comprises two layers of material of the higher friction section 232 including a top layer 240a that is folded over a bottom layer 240b. In other words, in each fold 236, the material of the higher friction section 232 is folded over itself once.

In other examples, the material of the higher friction section 232 can be folded over itself more than once or can be “bunched” within a fold. For example, FIG. 3B illustrates a fold 236ꞌ including a first portion 236aꞌ and a second portion 236bꞌ, where each portion comprises multiple layers of material of the higher friction section 232 including multiple top layers 242a folded over a bottom layer 242b. In the illustrated example of FIG. 3B, the material of the higher friction section 232 is folded over itself three times at regular intervals or in sections of approximately the same size in an accordion-type configuration. In some examples, the material of the higher friction section 232 can be folded over itself more or fewer times within the fold 236bꞌ (for example, two times or four times). In some examples, the material of the higher friction section 232 can be bunched within the fold 236bꞌ such that the folding over the material is irregular or has a more scrunched configuration relative to the example of FIG. 3B.

It will be appreciated that other fold patterns can be utilized to form folds in the outer skirt 218. For example, the higher friction sections 232 can be in a rolled configuration within the interior folds 236. It will also be appreciated that the exemplary fold configurations of FIG. 3A and 3B are shown in a relatively “loose” state with spaces between adjacent layers of each fold in order to illustrate the fold patterns. In use, in some examples, the fold patterns can be formed in a “tighter” configuration so that the outer skirt 218 is more compact on the outer surface of the frame 212 than is shown in the illustrations and adjacent layers (for example, adjacent layers 240a and 240b or adjacent layers 242a and 242b) are in contact with each other when the prosthetic valve is in the radially compressed state for delivery.

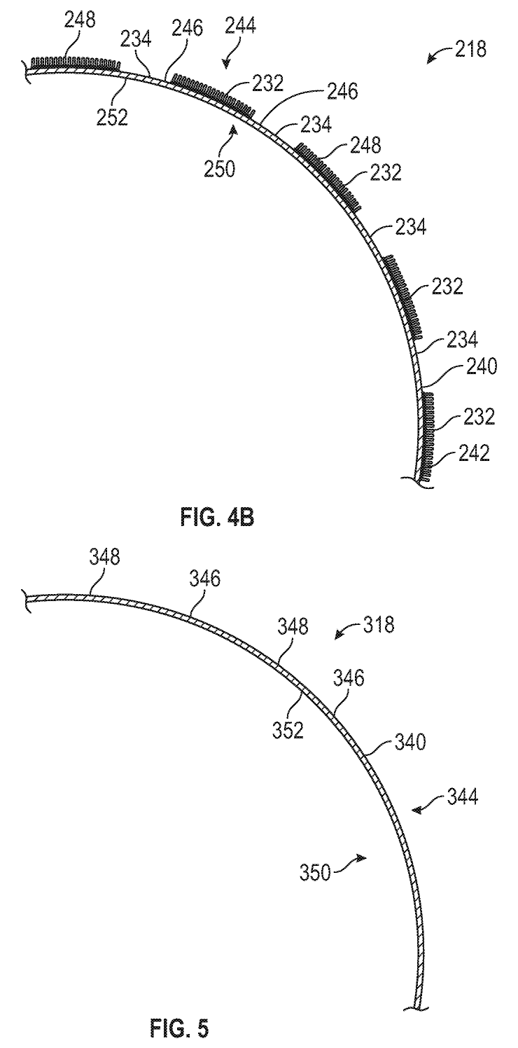

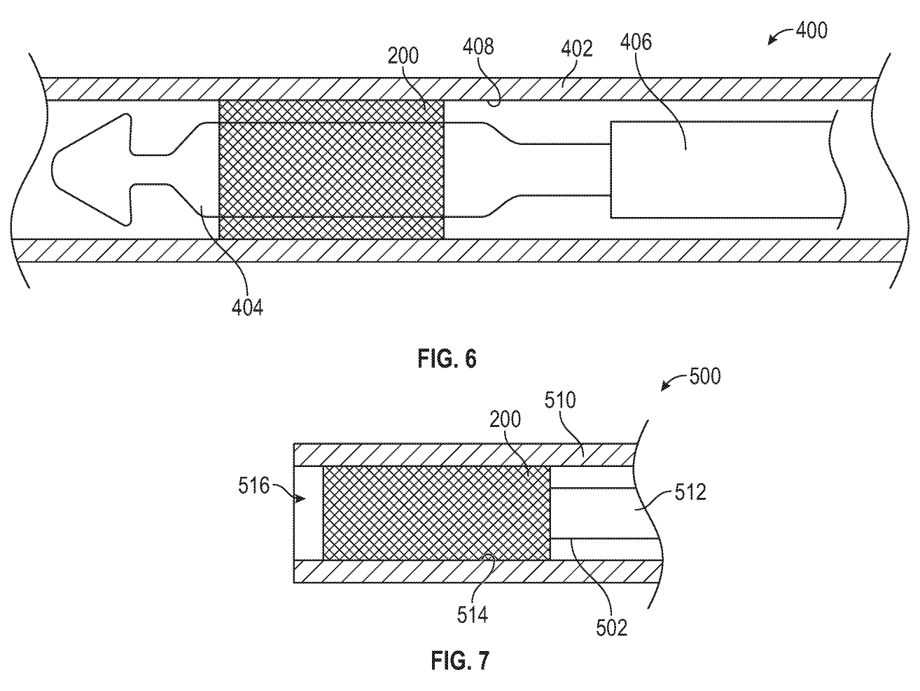

As discussed above, for any of the foregoing exemplary fold patterns, the higher friction sections 232 can be completely or partially covered or shielded by the lower friction sections 234 while the prosthetic valve 200 is radially compressed. Thus, in some examples, the lower friction sections 234 can form at least the majority of the outer surface of the outer skirt while the prosthetic valve 200 is radially compressed. In some examples, the higher friction sections 232 can be completely covered by the lower friction sections 234 while the prosthetic valve 200 is radially compressed, and the lower friction sections 234 can form the entirety of the outer surface of the outer skirt when the prosthetic heart valve 200 is radially compressed. These exemplary configurations can prevent or minimize contact between the higher friction sections 232 and components of a delivery apparatus and/or an introducer sheath during transcatheter delivery of the radially compressed prosthetic valve 200. In other words, in such examples, the lower friction sections 234 contact components of a delivery apparatus and/or an introducer sheath during transcatheter delivery of the radially compressed prosthetic valve 200 and enable smoother or easier (for example, requiring a less axial force exerted thereon) movement of the radially compressed prosthetic valve relative to the delivery apparatus or introducer sheath components (discussed further below with reference to FIGS. 6 and 7)

When the prosthetic valve 200 is deployed from a delivery apparatus, the prosthetic valve can be transitioned from the radially compressed state (FIGS. 3A-3B) to the radially expanded state (FIGS. 4A-4B). When the prosthetic heart valve transitions from the radially compressed state to the radially expanded state, each of the folds 236, 238 (formed by the higher friction sections 232 and the lower friction sections 234) can unfold. In some examples, when the prosthetic heart valve is in the radially expanded state, at least a majority of the total surface area of the higher friction sections 234 is uncovered by the lower friction sections 234. In some examples, as can be seen in FIG. 4A, when the prosthetic heart valve is in the radially expanded state, the higher friction sections 232 can be completely uncovered by the lower friction sections 234. In other words, the higher friction sections 232 can be released or uncovered or exposed from the lower friction sections 234. Thus, in the radially expanded state of the prosthetic heart valve 200, an outer surface of the prosthetic valve can include the axially extending higher frictions sections 232 and lower frictions sections 234 in an alternating arrangement around the circumference of the radially expanded prosthetic valve 200. Further, the higher friction sections 232 can form 60% to 80 % of the total surface area of the exterior surface of the outer skirt on the exterior surface of the radially expanded prosthetic valve.

As noted above, the material of the higher friction sections 232 can encourage tissue ingrowth. Accordingly, when the prosthetic heart valve 200 is radially expanded within a native heart valve, the higher friction 232 can encourage ingrowth of tissue at or surrounding the native heart valve and/or can help establish a seal with the surrounding native annulus to minimize or prevent paravalvular leakage (PVL).

In some examples, the outer skirt 218 can further comprise one or more surfaces having a treatment or coating, or can have a surface treatment layer disposed thereon. In some examples, the treated or coated surfaces or layers can be an agent-eluting surface or layer, such as, for example, a drug or lubricant-eluting surface or layer. For example, as shown in FIG. 4B, an exterior side 244 of the outer skirt 218 can include coated or treated surfaces 246 at one or more of the lower friction sections 234 and coated or treated surfaces 248 at one or more of the higher friction sections 232, and an interior side 250 of the outer skirt 218 can include a coated or treated surface 252 extending over or on at least a portion of the interior surface. In other examples, an outer skirt can exclude one or more of the coated or treated surfaces shown in FIG. 4B. For example, an outer skirt can include just one of the coated or treated surfaces 246, 248, 252. In some examples, the treated or coated surfaces 246, 248, 252 can comprise treated or coated sutures configured to elute an agent or substance (for example, a drug or a lubricant therefrom).

In some examples, each of the coated or treated surfaces 246, 248, 252 can comprise a different type of coating or treatment relative to the other coated or treated surfaces. For example, the coated or treated surfaces 246 can comprise a hydrophilic or lubricous coating or surface treatment. In some examples, the coated or treated surface 246 can be configured to elute a hydrophilic or lubricious agent therefrom. In some examples, the coated or treated surface 246 comprises polyethylene glycol (PEG). In some examples, PEG is elutable from the outer skirt upon contact with fluid (such as, for example, blood). In some examples, the higher friction sections 232 are free of the hydrophilic or lubricous coating or surface treatment. In some examples, the hydrophilic or lubricous coating or surface treatment can create a slippery or lubricated surface (with a reduced coefficient of friction) at the lower friction sections 234 of the outer skirt 218. In some examples, during delivery of the radially compressed prosthetic valve, the hydrophilic or lubricous coating or surface treatment can assist in or enable smoother or easier (for example, requiring a less axial force exerted thereon) movement of the radially compressed prosthetic valve relative to the delivery apparatus or introducer sheath components (discussed further below with respect to FIGS. 6 and 7).

In some examples, the coated or treated surfaces 248 can comprise a prothrombogenic surface treatment or coating. In some examples, the prothrombogenic surface treatment or coating can comprise biological molecules, such as growth factors, or can comprise other treatments or coatings for encouraging tissue ingrowth and/or encouraging thrombosis. In some examples, the coated or treated surface 248 can be configured to elute a prothrombogenic agent therefrom (for example, biological molecules, such as growth factors). In some examples, a prothrombogenic agent is elutable from the prothrombogenic surface treatment or coating upon contact with fluid (for example, blood). In some examples, the lower friction sections 232 are free of the prothrombogenic surface treatment or coating. In some examples, the prothrombogenic surface treatment or coating can enable, encourage, or increase tissue ingrowth at the higher friction sections 232 of the outer skirt 218. In some examples, after deployment of the prosthetic valve 200 at an implantation site, the prothrombogenic surface treatment or coating can assist in, encourage, or enable stabilization a position of the implanted prosthetic heart valve, limit movement or dislodgement of the implanted prosthetic heart valve, and/or form of a seal between the native anatomy and the prosthetic heart valve.

In some examples, the coated or treated surface 252 can comprise an antithrombogenic surface treatment or coating. In some examples, the antithrombogenic surface treatment or coating can comprise a hydrogel coating, heparin coating, ticlopidine coating, phosphorylcholine coating, or phenox hydrophilic polymer coating, or other treatment of coating for discouraging tissue ingrowth and/or discouraging thrombosis or pannus formation. In some examples, the coated or treated surface 252 can be configured to elute an antithrombogenic agent therefrom. In some examples, the antithrombogenic agent (for example, heparin, ticlopidine, or phosphorylcholine) is elutable from the antithrombogenic surface treatment or coating upon contact with fluid (for example, blood). In some examples, after implantation of the prosthetic valve 200, the coated or treated surface 252 can be configured to release antithrombotic or antiproliferative drugs, to resist thrombus or pannus formation in a "neo-sinus" region defined between the outer skirt 218 and leaflets of the valve structure of the prosthetic valve 200. In some examples, after implantation of the prosthetic valve 200, the antithrombogenic surface treatment or coating can discourage, limit, or prevent blocking or occlusion of the interior of the prosthetic valve (such as at, for example, the valve structure).

As discussed above, in some examples, the base layer 240 can be an impermeable or semi-impermeable layer that can limit or prevent elution or permeation of substances or agents thereacross. For example, the base layer can prevent or limit agents eluted from the treated or coated surfaces 246, 248 from being released or eluted to the interior side 250 of the outer skirt 218, and can prevent or limit agents eluted from the treated or coated surface 252 from being released or eluted to the exterior side 244 of the outer skirt 218. In other words, the outer skirt 218 can include an impermeable or semi-impermeable layer (that is, the base layer), an interior agent-eluting layer on the interior surface of the outer skirt, and an exterior agent-eluting layer on the exterior surface of the outer skirt. As discussed above, in some examples the interior agent-eluting layer can include an antithrombogenic agent-eluting layer, and the exterior agent-eluting layer can include a prothrombogenic agent-eluting layer and/or a lubricious agent-eluting layer.

In some examples, the base layer 240 can comprise PTFE, a thin layer of PET, TPU, or other impermeable or semi-impermeable materials. In examples where the base layer 240 is a semi-impermeable layer, the material of the base layer can be selected to limit or prevent elution or permeation of a first substance (for example, a large molecule drug) and can be selected to allow or enable elution or permeation of a second substance (for example, a small molecule drug or blood).

In some examples, the treated or coated surfaces 246, 248, 252 can be configured for time-release or elution or controlled-release or elution of an agent or substance therefrom. In some examples, one or more of the treated or coated surfaces can be configured for a slower time-release relative to others of the treated or coated surfaces (which are configured for faster time-release). In some examples, the treated or coated surfaces 246, 248, 252 can be designed or configured to release the corresponding agent (for example, drug or lubricant) at different phases or for different time durations over the processes of delivery, implantation, and normal functioning of the prosthetic valve.

For example, the coated or treated surfaces 246 comprising a hydrophilic or lubricous coating or surface treatment can be relatively faster-acting or quicker-time-release or shorter-duration release surfaces in order to lubricate the prosthetic valve during delivery to the implantation site and to stop or limit lubrication after implantation of the prosthetic valve. In another example, the coated or treated surface 252 comprising an antithrombogenic surface treatment or coating can be a relatively slower-acting or slower-time-release or longer-duration release surface in order to prevent or limit thrombosis and/or pannus formation over the implantation lifetime (or at least a portion thereof) and normal functioning of the prosthetic valve. In yet another example, the coated or treated surfaces 248 comprising a prothrombogenic surface treatment or coating can be slower-acting or slower-time release or longer-duration release surfaces relative to the hydrophilic or lubricous surfaces 246 and/or can be a faster-acting or quicker-time release or shorter-duration release surfaces relative to the antithrombogenic surface 252 in order to encourage tissue in-growth and stabilize the prosthetic valve shortly after its implantation or deployment within a native heart valve.

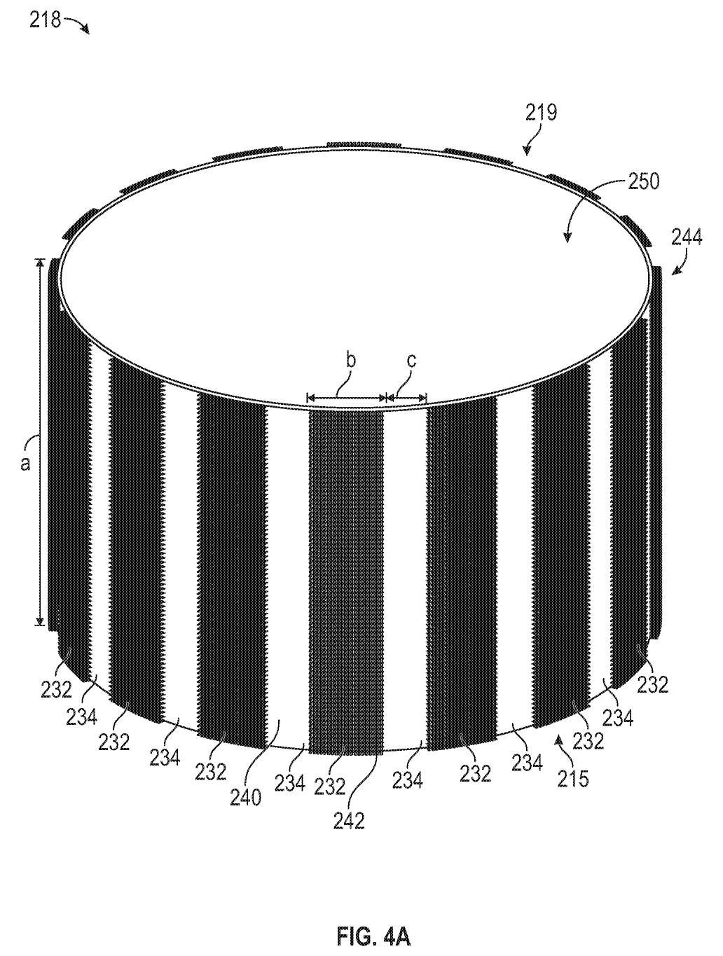

FIG. 5 illustrates another exemplary outer skirt 318 that can be utilized with the prosthetic valves disclosed herein. The outer skirt 318 can comprise a base layer 340 (similar to the base layer 240 discussed above) without additional textile material (such as, for example, a plush or pile material) attached thereto.

The outer skirt 318 can further comprise one or more surfaces having a treatment or coating, such as treated or coated surfaces 346, 348 on an exterior surface 344 of the outer skirt and a treated or coated surface 352 on an interior surface 350 of the outer skirt. In some examples, the treated or coated surfaces 346, 348, 352 can have one or more features respectively corresponding the treated or coated surfaces 246, 248, 252 discussed above. The treated surfaces 346, 348 can correspond in size and shape to the lower friction surfaces 234 and the higher friction surfaces 232, respectively.

For example, the treated or coated surfaces 346 include a hydrophilic or lubricious treatment or coating, while the treated or coated surfaces 348 can be free of a hydrophilic or lubricious treatment or coating. In such examples, the treated or coated surfaces 346 comprise lower friction sections relative to the treated or coated surfaces 348, and the treated or coated surfaces 348 (higher friction sections) can be inwardly folded and covered by the treated or coated surfaces 346 (lower friction sections) when the prosthetic valve is in a radially compressed state (for example, in manner similar to the folding patterns illustrated in FIGS. 3A-3B) for delivery of the prosthetic valve to an implantation site. When the prosthetic valve is radially expanded, the treated or coated surfaces 348 including a prothrombogenic agent can be exposed on the exterior surface of the valve and promote tissue ingrowth into the outer skirt 318.

In some examples, a skirt 318 can comprises a base layer 340, which can comprise any of various textiles described above (such as a woven fabric) or a film layer, with sections on the outer surface of the skirt corresponding in size and shape with sections 234 coated with a hydrophilic or lubricious treatment or coating and sections on the outer surface corresponding in size and shape with sections 232 that are devoid of the hydrophilic or lubricious treatment or coating. The sections without the hydrophilic or lubricious treatment or coating need not include any surface treatments or coatings and the inner surface of the skirt need not include any surface treats or coatings.

In some examples, a skirt 318 can comprises a base layer 340 and the inner surface is coated or treated with an anti-thrombogenic agent (such as any of those described above) and the outer surface is coated or treated with a prothrombogenic agent (such as any of those described above). The base layer 340 preferably comprises a material or construction that is impermeable or semi-impermeable as disclosed above to prevent or minimize the agents from migrating through the skirt from the outer surface to the inner surface, or vice versa.

In other examples, the outer skirt 318 can exclude one or more of the treated or coated surfaces 346, 348, 352. In other examples, the treated or coated surfaces 346, 348 can be coextensive and layered over the exterior surface 344 of the outer skirt 318 rather than being disposed in discrete sections. In such examples, the lubricious coating or layer can be a quicker-release or shorter-duration release coating that is an exterior layer relative to an underlying prothrombogenic coating or layer.

In some examples, sutures can be woven or otherwise attached to the outer skirt 318 and exposed portions of the sutures on the inner surface of the skirt and/or the outer surface of the skirt can have any of the coatings or agents described above for surfaces 246, 248, 252, 346, 348, or 352. For example, the sutures used to secure the outer skirt to the frame can have such coatings or agents.

An outer skirt having a prothrombogenic coating or agent on its outer surface can be particularly helpful for improving sealing with the surrounding anatomy at an implantation site of a patient suffering from aortic insufficiency (AI). For example, the prothrombogenic coating or agent can improve sealing with the native aortic annulus and/or native aortic leaflets. A prosthetic valve having such an outer skirt also can include an anchoring frame (in addition to frame 212) to assist in anchoring the prosthetic valve within an insufficient aortic valve, such as disclosed in WIPO Publication No. WO 2021/242948, which is incorporated herein by reference.

Turning to FIGS. 6 and 7, exemplary implant delivery systems 400 and 500 including the prosthetic valve 200 are shown and described. As shown in the exemplary delivery system 400 of FIG. 6, in some examples, the prosthetic heart valve 200 can be sized and shaped (when in the radially compressed state) to be inserted through an introducer sheath 402 for transcatheter delivery of the prosthetic valve 200 to an implantation site. In some examples, the radially compressed prosthetic valve 200 can be a balloon-expandable prosthetic valve and can be mounted over or around an inflatable balloon 404 a delivery apparatus 406. The delivery apparatus 406 is a schematic representation of a delivery apparatus (for example, delivery apparatus 100) used for implanting a balloon-expandable prosthetic valve. The outer skirt 218 (not shown in FIG. 6) can be folded, such as shown in FIG. 3A or FIG. 3B, so that the lower friction surfaces 234 are exposed. As the delivery apparatus 406 and the radially compressed prosthetic valve 200 are advanced through the introducer sheath 402, an exterior surface of the folded outer skirt 218 can contact and slide against an interior surface 408 of the introducer sheath 402. .

As discussed above, when the prosthetic valve 200 is in the radially compressed state, a majority or all of the exterior surface of the skirt 218 is formed by lower friction sections 234 (that is, the exterior folds 238) and a majority or an entirety of the higher friction sections 232 (that is, the interior folds 236) are shielded or covered by the lower friction sections 234. Thus, friction between the exterior surface 254 of the outer skirt 218 (forming at least a portion of the outer surface of the prosthetic valve 200 valve) and the interior surface 408 of the introducer sheath 402 can be reduced relative to a prosthetic valve including an outer skirt comprising only a higher friction material or having portions of higher friction material disposed on the exterior surface of the prosthetic valve while it is radially compressed. In examples including a hydrophilic or lubricous coating or surface treatment on the outer skirt 218, the hydrophilic or lubricous coating or surface treatment can lubricate the exterior surface of the outer skirt 218 (forming at least a portion of the outer surface of the prosthetic valve 200 valve) for advancement of the prosthetic valve 200 through the introducer sheath 402. In such examples, friction between the exterior surface of the outer skirt 218 (forming at least a portion of the outer surface of the prosthetic valve 200 valve) and the interior surface 408 of the introducer sheath 402 can be reduced relative to a prosthetic valve including an outer skirt lacking hydrophilic or lubricous coating or surface treatment. Accordingly, in any of the foregoing examples, less pushing force may be required on the delivery apparatus 406 for advancement of the prosthetic valve 200 through the introducer sheath 402 and/or movement of the prosthetic valve 200 through the introducer sheath 402 can be smoother and/or more predictable.

As shown in the exemplary delivery system 500 of FIG. 7, in some examples, when in a radially compressed state, the prosthetic heart valve 200 (when in the radially compressed state) can be sized and shaped to be disposed within a capsule or delivery sheath 510 of a delivery apparatus 502 for transcatheter delivery of the prosthetic heart valve to an implantation site. In some examples, the radially compressed prosthetic valve 200 can be a self-expandable prosthetic valve mounted or coupled to a distal end of a shaft 512 of the delivery apparatus 502 and retained in the radially compressed state by the capsule 510. The delivery apparatus 502 is a schematic representation of a delivery apparatus used to implant a self-expandable prosthetic valve. The radially compressed prosthetic valve 200 mounted on the delivery shaft 512 and retained with the capsule 510 can be inserted through a patient’s vasculature to the implantation site. The self-expandable prosthetic valve 200 can be configured to radially expand on its own upon either removal (withdrawal) of the capsule 510 covering the radially compressed prosthetic heart valve or forward advancement of the prosthetic valve through a distal opening 516 of the capsule 510. As the radially compressed prosthetic valve 200 is deployed from the capsule 510, the exterior surface of the folded outer skirt 218 can contact and slide against an interior surface 514 of the capsule 510.

As discussed above, when the prosthetic valve 200 is in the radially compressed state, a majority or all of the exterior surface of the skirt 218 is formed by lower friction sections 234 (that is, the exterior folds 238) and a majority or an entirety of the higher friction sections 232 (that is, the interior folds 236) are shielded or covered by the lower friction sections 234. Thus, friction between the exterior surface of the outer skirt 218 (forming at least a portion of the exterior surface of the prosthetic valve 200 valve) and the interior surface 514 of the capsule 510 can be reduced relative to a prosthetic valve including an outer skirt comprising only a higher friction material or having portions of higher friction material disposed on the exterior surface of the prosthetic valve while it is radially compressed. In examples including a hydrophilic or lubricous coating or surface treatment on the outer skirt 218, the hydrophilic or lubricous coating or surface treatment can lubricate the exterior surface of the outer skirt 218 for movement of the radially compressed prosthetic heart valve 200 relative to the capsule 510. In such examples, friction between the exterior surface the outer skirt 218 (forming at least a portion of the prosthetic valve 200 valve) and the interior surface 514 of the capsule 510 can be reduced relative to a prosthetic valve including an outer skirt lacking hydrophilic or lubricous coating or surface treatment. Accordingly, in any of the foregoing examples, less force may be required on the shaft 512 or on the capsule 510 for movement of the prosthetic valve 200 relative to the capsule 510 and/or deployment of the prosthetic valve 200 from the capsule 510 can be smoother and/or more predictable.

### Delivery Techniques

For implanting a prosthetic valve (such as, for example, the prosthetic valves 10, 200 disclosed herein) within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve (such as, for example, the prosthetic valves 10, 200 disclosed herein) within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve (such as, for example, the prosthetic valves 10, 200 disclosed herein) within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve, comprising: an annular frame having an inflow end and an outflow end, wherein the annular frame is radially expandable for transitioning the prosthetic heart valve from a radially compressed state to a radially expanded state; a leaflet structure positioned within an interior of the frame; and an outer skirt positioned on an exterior of the frame, wherein an exterior surface of the outer skirt comprises a plurality of lower friction sections and a plurality of higher friction sections; wherein, in the radially compressed state of the prosthetic heart valve, the higher friction sections form a plurality of interior folds of the outer skirt and the lower friction sections form a plurality of exterior folds of the outer skirt that extend at least partially over the interior folds such that at least a portion of a total surface area of the higher friction sections is covered by the lower friction sections, and in the radially expanded state of the prosthetic heart valve, a lesser extent of the total surface area of the higher friction sections is covered by the lower friction sections.

Example 2. The prosthetic heart valve of any example disclosed herein, particularly example 1, wherein the lower friction sections and the higher friction sections have an alternating arrangement around a circumference of the outer skirt.

Example 3. The prosthetic heart valve of any example disclosed herein, particularly examples 1 or 2, wherein the higher friction sections and the lower friction sections extend axially from an inflow end of the outer skirt to an outflow end of the outer skirt.

Example 4. The prosthetic heart valve of any example disclosed herein, particularly examples 1-3, wherein each of the higher friction sections comprises a textured material.

Example 5. The prosthetic heart valve of any example disclosed herein, particularly example 4, wherein the textured material comprises a plurality of fibers that extend radially outward from the exterior surface of the outer skirt.

Example 6. The prosthetic heart valve of any example disclosed herein, particularly examples 4 or 5, wherein the textured material comprises at least one of a pile material, a plush material, or a fringed material.

Example 7. The prosthetic heart valve of any example disclosed herein, particularly examples 4-6, wherein each of the lower friction sections comprises a smoother material relative to the textured material.

Example 8. The prosthetic heart valve of any example disclosed herein, particularly examples 4-7, wherein the outer skirt comprises a base layer, wherein the textured material is attached to or formed on the base layer at the higher friction sections, and wherein the lower friction sections comprise exposed portions of the base layer that are devoid of the textured material.

Example 9. The prosthetic heart valve any example disclosed herein, particularly example 8, wherein the base layer comprises an impermeable or semi-impermeable material.

Example 10. The prosthetic heart valve any example disclosed herein, particularly example 9, wherein an inner surface of the outer skirt comprises an antithrombogenic surface treatment or coating.

Example 11. The prosthetic heart valve of any example disclosed herein, particularly examples 9 or 10, wherein the exterior surface of the outer skirt comprises a prothrombogenic surface treatment or coating.

Example 12. The prosthetic heart valve of any example disclosed herein, particularly examples 9-11, wherein the antithrombogenic surface treatment or coating comprises a slower-release treatment or coating, and wherein the prothrombogenic surface treatment or coating comprises a faster-release treatment or coating.

Example 13. The prosthetic heart valve of any example disclosed herein, particularly examples 9-12, wherein an antithrombogenic agent is elutable from the antithrombogenic surface treatment or coating upon contact with fluid.

Example 14. The prosthetic heart valve of any example disclosed herein, particularly examples 9-13, wherein a prothrombogenic agent is elutable from the prothrombogenic surface treatment or coating upon contact with fluid.

Example 15. The prosthetic heart valve of any example disclosed herein, particularly examples 1-14, wherein the lower friction sections comprise a hydrophilic surface treatment or coating.

Example 16. The prosthetic heart valve of any example disclosed herein, particularly example 15, wherein the hydrophilic surface treatment or coating comprises polyethylene glycol (PEG).

Example 17. The prosthetic heart valve of any example disclosed herein, particularly example 16, wherein the polyethylene glycol (PEG) is elutable from the outer skirt upon contact with fluid.

Example 18. The prosthetic heart valve of any example disclosed herein, particularly examples 15-17, wherein the higher friction sections are free of the hydrophilic surface treatment or coating.

Example 19. The prosthetic heart valve of any example disclosed herein, particularly examples 15-18, wherein, when in a radially compressed state, the prosthetic heart valve is sized and shaped to be disposed within a capsule of a delivery apparatus for transcatheter delivery of the prosthetic heart valve in the radially compressed state; and wherein, when the prosthetic heart valve is at or proximate a delivery site during transcatheter delivery thereof, the hydrophilic surface treatment or coating lubricates the prosthetic heart valve for advancement of the prosthetic heart valve from the capsule of the delivery apparatus.

Example 20. The prosthetic heart valve of any example disclosed herein, particularly examples 15-18, wherein, when in a radially compressed state, the prosthetic heart valve is sized and shaped to be inserted through an introducer sheath for transcatheter delivery of the prosthetic heart valve in the radially compressed state; and wherein, when the prosthetic heart valve is inserted through the introducer sheath during transcatheter delivery thereof, the hydrophilic surface treatment or coating lubricates the prosthetic heart valve for advancement of the prosthetic heart valve through the introducer sheath.

Example 21. The prosthetic heart valve of any example disclosed herein, particularly examples 1-20, wherein each of the higher friction sections has a greater surface area than each of the lower friction sections.

Example 22. The prosthetic heart valve of any examples disclosed herein, particularly examples 1-21, wherein the total surface area of the higher friction sections is greater than a total surface area of the lower friction sections.

Example 23. The prosthetic heart valve of any example disclosed herein, particularly example 22, wherein a ratio of a total surface area of the lower friction sections relative to a total surface area of higher friction sections is in a range of 0.2-0.4.

Example 24. The prosthetic heart valve of any example disclosed herein, particularly examples 1-23, wherein, when the prosthetic heart valve is in the radially compressed state, at least a majority of the total surface area of the higher friction sections is covered by the lower friction sections, and wherein, when the prosthetic heart valve is in the radially expanded state, at least a majority of the total surface area of the higher friction sections is uncovered by the lower friction sections.

Example 25. The prosthetic heart valve of any example disclosed herein, particularly example 24, wherein, when the prosthetic heart valve is in the radially compressed state, the higher friction sections are completely covered by the lower friction sections, and wherein, when the prosthetic heart valve is in the radially expanded state, the higher friction sections are completely uncovered by the lower friction sections.

Example 26. The prosthetic heart valve of any example disclosed herein, particularly examples 1-25, wherein, when the prosthetic heart valve transitions from the radially compressed state to the radially expanded state, the higher friction sections and the lower friction sections unfold.

Example 27. A prosthetic heart valve, comprising: an annular frame having an inflow end and an outflow end, wherein the annular frame is radially expandable for transitioning the prosthetic heart valve from a radially compressed state to a radially expanded state; a leaflet structure positioned within an interior of the frame; and an outer skirt positioned on an exterior of the frame, wherein an exterior surface of the outer skirt comprises a plurality of lower friction sections and a plurality of higher friction sections; wherein, in the radially compressed state of the prosthetic heart valve, the higher friction sections form a plurality of interior folds of the outer skirt and the lower friction sections extend at least partially over the interior folds; and wherein, when the prosthetic heart valve transitions from the radially compressed state to the radially expanded state, the higher friction sections unfold such that a greater portion of the higher friction sections are uncovered from the lower friction sections relative to when the prosthetic heart valve is in the radially compressed state.

Example 28. The prosthetic heart valve of any example disclosed herein, particularly example 27, wherein the lower friction sections and the higher friction sections have an alternating arrangement around a circumference of the outer skirt.

Example 29. The prosthetic heart valve of any example disclosed herein, particularly examples 27 or 28, wherein the lower friction sections and the higher friction sections extend axially from an inflow end of the outer skirt to an outflow end of the outer skirt.

Example 30. The prosthetic heart valve of any example disclosed herein, particularly examples 27-29, wherein each of the higher friction sections comprises a textured material comprising a plurality of fibers that extend radially outward from the exterior surface of the outer skirt.

Example 31. The prosthetic heart valve of any example disclosed herein, particularly example 30, wherein the textured material comprises at least one of a pile material, a plush material, or a fringed material.

Example 32. The prosthetic heart valve of any example disclosed herein, particularly examples 27-31, wherein each of the lower friction sections comprises a smoother material relative to the higher friction sections.

Example 33. The prosthetic heart valve of any example disclosed herein, particularly examples 27-32, wherein the outer skirt further comprises an impermeable or semi-impermeable layer and at least one surface treatment or coating on the exterior surface of the outer skirt.

Example 34. The prosthetic heart valve of any example disclosed herein, particularly example 33, wherein at least the lower friction sections of the outer skirt comprise a first surface treatment or coating, the first surface treatment or coating comprising a lubricious agent elutable from the lower friction sections of the exterior surface of the outer skirt.

Example 35. The prosthetic heart valve of any example disclosed herein, particularly examples 33 or 34, wherein at least the higher friction sections of the outer skirt comprise a second surface treatment or coating, the second surface treatment or coating comprising an antithrombogenic agent elutable from the higher friction sections of the exterior surface of the outer skirt.

Example 36. The prosthetic heart valve of any example disclosed herein, particularly example 33-35, wherein the outer skirt further comprises a third surface treatment or coating on an inner surface of the outer skirt, the third surface treatment or coating comprising an antithrombogenic agent elutable from the inner surface of the outer skirt.

Example 37. The prosthetic heart valve of any example disclosed herein, particularly example 36, wherein the third surface treatment or coating on the inner surface of the outer skirt comprises a slower-release surface treatment or coating relative to the at least one surface treatment or coating on the exterior surface of the outer skirt.

Example 38. A prosthetic heart valve, comprising: an annular frame having an inflow end and an outflow end; a leaflet structure positioned within an interior of the frame; and an outer skirt positioned on an exterior of the frame and secured to the frame, wherein the outer skirt comprises an impermeable or semi-impermeable layer, an interior agent-eluting layer, and an exterior agent-eluting layer.

Example 39. The prosthetic heart valve of any example disclosed herein, particularly example 38, wherein the interior agent-eluting layer comprises an antithrombogenic agent-eluting layer.

Example 40. The prosthetic heart valve of any example disclosed herein, particularly examples 38 or 39, wherein the exterior agent-eluting layer comprises a prothrombogenic agent-eluting layer.

Example 41. The prosthetic heart valve of any example disclosed herein, particularly examples 38-40, wherein the exterior agent-eluting layer comprises a lubricious agent-eluting layer.

Example 42. The prosthetic heart valve of any example disclosed herein, particularly example 41, wherein the exterior agent-eluting layer comprises polyethylene glycol (PEG), and the PEG is elutable from the exterior agent-eluting layer upon contact with fluid.

Example 43. The prosthetic heart valve of any example disclosed herein, particularly examples 38-42, wherein the interior agent-eluting layer is slower substance-eluting layer relative to the exterior agent-eluting layer.

Example 44. A prosthetic heart valve comprising: an annular frame having an inflow end and an outflow end, wherein the annular frame is radially expandable for transitioning the prosthetic heart valve from a radially compressed state to a radially expanded state; a leaflet structure positioned within an interior of the frame; and an outer skirt positioned on an exterior of the annular frame and secured to the annular frame, the outer skirt comprising a plurality of first sections and a plurality of second sections, wherein the plurality of first sections have a first coefficient of friction, wherein the plurality of second sections have a second coefficient of friction, and wherein the first coefficient of friction is greater than the second coefficient of friction; wherein a ratio of an exposed surface area of the first sections to an exposed surface area of the second sections is greater in the radially expanded state than in the radially compressed state of the prosthetic heart valve.

Example 45. The prosthetic heart valve of any example disclosed herein, particularly example 44, wherein, in the radially compressed state of the prosthetic heart valve, a portion of each of the first sections forms at least one overlap and covers another portion of the respective first section.

Example 46. The prosthetic heart valve of any example disclosed herein, particularly examples 44 or 45, wherein the first sections and the second sections extend axially from an inflow end of the outer skirt to an outflow end of the outer skirt and have an alternating arrangement around a circumference of the outer skirt.

Example 47. A prosthetic heart valve according to any example herein, wherein the prosthetic heart valve is sterilized.

Example 48. A method of sterilizing any of the prosthetic heart valves described herein.

The features described herein with regard to any example can be combined with other features described in any one or more of the other examples, unless otherwise stated. For example, any one or more of the features of one prosthetic heart valve can be combined with any one or more features of another prosthetic heart valve. As another example, any one or more features of one outer skirt can be combined with any one or more features of another outer skirt.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve, comprising:
an annular frame having an inflow end and an outflow end, wherein the annular frame is radially expandable for transitioning the prosthetic heart valve from a radially compressed state to a radially expanded state;
a leaflet structure positioned within an interior of the frame; and
an outer skirt positioned on an exterior of the frame, wherein an exterior surface of the outer skirt comprises a plurality of lower friction sections and a plurality of higher friction sections;
wherein the lower friction sections and the higher friction sections extend axially from an inflow end of the outer skirt to an outflow end of the outer skirt.

2. The prosthetic heart valve of claim 1, wherein, in the radially compressed state of the prosthetic heart valve, the higher friction sections form a plurality of interior folds of the outer skirt and the lower friction sections extend at least partially over the interior folds.

3. The prosthetic heart valve of claim 2, wherein, when the prosthetic heart valve transitions from the radially compressed state to the radially expanded state, the higher friction sections unfold such that a greater portion of the higher friction sections are uncovered from the lower friction sections relative to when the prosthetic heart valve is in the radially compressed state.

4. The prosthetic heart valve of claim 1, wherein the lower friction sections and the higher friction sections have an alternating arrangement around a circumference of the outer skirt.

5. The prosthetic heart valve of claim 1, wherein each of the higher friction sections comprises a textured material comprising a plurality of fibers that extend radially outward from the exterior surface of the outer skirt.

6. The prosthetic heart valve of claim 5, wherein the textured material comprises at least one of a pile material, a plush material, or a fringed material.

7. The prosthetic heart valve of claim 1, wherein each of the lower friction sections comprises a smoother material relative to the higher friction sections.

8. The prosthetic heart valve of claim 1, wherein the outer skirt further comprises an impermeable or semi-impermeable layer and at least one surface treatment or coating on the exterior surface of the outer skirt.

9. The prosthetic heart valve of claim 8, wherein the surface treatment or coating comprises a lubricious agent elutable from the lower friction sections of the exterior surface of the outer skirt.

10. The prosthetic heart valve of claim 8, wherein the surface treatment or coating comprises an antithrombogenic agent elutable from the higher friction sections of the exterior surface of the outer skirt.

11. The prosthetic heart valve of claim 1, wherein the outer skirt further comprises a surface treatment or coating on an inner surface of the outer skirt, which comprises an antithrombogenic agent elutable from the inner surface of the outer skirt.

12. The prosthetic heart valve of claim 11, wherein the surface treatment or coating on the inner surface of the outer skirt comprises a slower-release surface treatment or coating relative to a surface treatment or coating on the exterior surface of the outer skirt.

13. A prosthetic heart valve, comprising:
an annular frame having an inflow end and an outflow end;
a leaflet structure positioned within an interior of the frame; and
an outer skirt positioned on an exterior of the frame and secured to the frame, wherein the outer skirt comprises an impermeable or semi-impermeable layer, an interior agent-eluting layer, and an exterior agent-eluting layer.

14. The prosthetic heart valve of claim 13, wherein the interior agent-eluting layer comprises an antithrombogenic agent-eluting layer.

15. The prosthetic heart valve of claim 13, wherein the exterior agent-eluting layer comprises a prothrombogenic agent-eluting layer.

16. The prosthetic heart valve of claim 13, wherein the exterior agent-eluting layer comprises a lubricious agent-eluting layer.

17. The prosthetic heart valve of claim 16, wherein the exterior agent-eluting layer comprises polyethylene glycol (PEG), and the PEG is elutable from the exterior agent-eluting layer upon contact with fluid.

18. The prosthetic heart valve of claim 13, wherein the interior agent-eluting layer is slower substance-eluting layer relative to the exterior agent-eluting layer.

19. A prosthetic heart valve comprising:
an annular frame having an inflow end and an outflow end, wherein the annular frame is radially expandable for transitioning the prosthetic heart valve from a radially compressed state to a radially expanded state;
a leaflet structure positioned within an interior of the frame; and
an outer skirt positioned on an exterior of the annular frame and secured to the annular frame, the outer skirt comprising a plurality of first sections and a plurality of second sections, wherein the plurality of first sections have a first coefficient of friction, wherein the plurality of second sections have a second coefficient of friction, and wherein the first coefficient of friction is greater than the second coefficient of friction;
wherein a ratio of an exposed surface area of the first sections to an exposed area of the second sections is greater in the radially expanded state than in the radially compressed state of the prosthetic heart valve.

20. The prosthetic heart valve of claim 19, wherein, in the radially compressed state of the prosthetic heart valve, a portion of each of the first sections forms at least one overlap and covers another portion of the respective first section.

21. The prosthetic heart valve of claim 20, wherein the first sections and the second sections extend axially from an inflow end of the outer skirt to an outflow end of the outer skirt and have an alternating arrangement around a circumference of the outer skirt.
